(19) United States
(12) Patent Application Publication
(10) Pub. No.: US 2023/0197262 A1
(43) Pub. Date: Jun. 22, 2023

(54) APPARATUS AND METHODS FOR PULMONARY MONITORING

(71) Applicant: THE REGENTS OF THE UNIVERSITY OF CALIFORNIA, Oakland, CA (US)

(72) Inventors: Mona ESKANDARI, Riverside, CA (US); Samaneh SATTARI, San Mateo, CA (US)

(73) Assignee: THE REGENTS OF THE UNIVERSITY OF CALIFORNIA, Oakland, CA (US)

(21) Appl. No.: 17/922,478

(22) PCT Filed: May 7, 2021
(86) PCT No.: PCT/US21/31306

§ 371 Date: Oct. 31, 2022

Related U.S. Application Data
(60) Provisional application No. 63/022,221, filed on May 8, 2020.

(51) Int. Cl. G16H 40/60; G16H 50/20

(52) U.S. Cl. CPC G16H 40/60; G16H 50/20; G16H 50/30

(57) ABSTRACT

An apparatus can monitor or assess a patient lung. The apparatus can include control circuitry configured to process a lung biomarker from patient data. The control circuitry can be configured to generate a lung index to characterize the patient lung to monitor or assess the patient lung.

### CLAIM OF PRIORITY

This patent application claims the benefit of priority of Mona Eskandari U.S. Provisional Patent Application Ser. No. 61,022,221, entitled “METHOD FOR PULMONARY MONITORING” filed on May 8, 2020, which is hereby incorporated by reference herein in its entirety.

### TECHNICAL FIELD

In the following description, for purposes of explanation, various details are set forth in order to provide a thorough understanding of some example embodiments. It will be apparent, however, to one skilled in the art that the present subject matter may be practiced without these specific details, or with slight alterations.

This document pertains generally, but not by way of limitation, to pulmonary monitoring.

### BACKGROUND

The present disclosure generally relates to a new diagnostic tool for lung health that harnesses a non-invasive measure of lung material properties. This can allow pulmonary monitoring that can be fast, routine, affordable, and as repeatable as taking a patient's pulse or measuring blood pressure.

Lung disease is a leading cause of death worldwide, and new threats are emerging from respiratory pandemics, vaping, and rising air pollution in many parts of the world. Current approaches to pulmonary examinations can be inaccurate, time-consuming, and inaccessible. As a result, lung health generally is not monitored unless symptomatic, and by then the damage can be permanent and degeneration can be irreversible. For example, COPD (chronic obstructive pulmonary disease) patients lose half their lung function before even receiving their first spirometry test.

All pulmonary function tests are currently based on measuring airflow during inhalation and exhalation. Drawbacks of traditional flow-measuring devices, such as spirometers (measuring the speed of air exhalation) and plethysmographs (a large system encasing the patient's entire body to record pressures and volumes), can include prolonged testing, inexact objective measures, and tedious technician training requirements. Further, the exams can be too lengthy for a typical doctor's office visit (20-30 minutes) or require the patient to be referred to a laboratory, by which time a symptom may have subsided. The testing protocols can be difficult to follow and repeat, especially for children; even adults can rarely reproduce their own results. More common are 30-second peak flow meter exams that provide little meaningful data. Even objective and insightful non-flow medical examinations based on imaging, such as lung CT scans (computerized tomography), notably used in the pressing COVID-19 outbreak, can be expensive and not widely accessible.

### SUMMARY

In consideration of the above issues, it would be desirable to provide a method that harnesses a fundamental scientific phenomenon, viscoelasticity response of the lung, to measure lung health quickly and routinely based on temporal pressure evolution, such as at least one of a change in respiratory tract pressure with time or a change in lung pressure with time, during a held inspiratory breath.

In addition, the method can be medically transformative, enabling early detection, differential diagnosis, and treatment assessment. As such, the method as disclosed herein has the potential to save lives, improve health outcomes, and save billions of dollars in diagnostic and treatment costs.

In an example, a method disclosed for pulmonary monitoring can include non-flow measurement of pressure evolution from an individual holding an inhaled breath.

In an example, the method can be used as a standard screening procedure, similar to other clinically pervasive and revolutionary devices such as blood pressure cuffs and glaucoma tonometry. In addition, the method can change the current pulmonary healthcare narrative by introducing a non-invasive, relatively fast, objective, and widely accessible assessment of lung health based on non-flow properties.

In an example, viscoelasticity can evaluate lung health using signature pressure-time (P-T) features, such as lung biomarkers, to classify normal and abnormal lung function, differentiate between types of abnormalities, and continuously monitor disease progression. Taken together, viscoelasticity can evaluate lung health using lung biomarkers to classify normal and abnormal lung function, differentiate between types of lung abnormalities, and continuously monitor lung disease progression.

In accordance with an exemplary embodiment, the whole-organ can be the entire lungs (i.e., right lung and left lung, or alternatively, only one lung if the patent or individual has only one lung) of a patient or individual.

In an example method, a patient can inhale and hold his or her breath as long as possible (for example, less than 30 seconds) to generate a pressure-time (P-T) curve, such as using a mouthpiece 210 acting as a real-time pressure gauge (manometer) interfacing with control circuitry, such as including a computing machine running a computer software to record and store measurements, for contemporaneous or later analysis. The P-T curve can characterize a change in pressure over time, such as a decrease of pressure over time, that can be analyzed by rheological models to generate a lung biomarker. A rheological model, such as conceptually consisting of discrete elements (springs and dashpots), can be used to curve-fit the P-T curve, such as an exponentially decaying P-T curve. A lung biomarker can include a signature feature of the P-T curve, such as at least one of an indication of a peak pressure, an indication of asymptotic pressure, an indication of fractional relaxation, an indication of a time-constant, an indication of degree of model non-linearity, or an indication of solid versus fluid proportional response (e.g., viscoelastic response). In an example, a lung biomarker can serve as an indication of patient lung health, such as a change in one or more lung biomarkers over time can indicate the risk for (or the presence of) a lung condition, such as in an asymptomatic patient.

In an example method, a patient can draw in and hold a breath, such as for a period of time to measure pressure evolution. Data obtained from the pressure evolution measurement can be applied to established rheological models to generate characteristic or signature features of a temporal pressure-versus-time (P-T) curve, such as to allow the a comparison of features of healthy control, such as a “normal” lung, to diseased states, such as an “abnormal” lung. Differences between signature features of healthy control data and diseased state data can be used, such as to detect the abnormal lung state. In an example, the method disclosed herein can also be extended to additional diseased states, such as to explore possible differential diagnostic capabilities or for disease progression monitoring.

In an example, when a single characteristic feature of viscoelasticity (e.g., percent pressure relaxation) can be compared between healthy tissue and dust-exposed tissue modeling asthma, the asthmatic model exhibits notably decreased fractional (or percent pressure) relaxation, such as to indicate the presence of a lung condition in the tissue (e.g., asthma). Additional features such as peak and asymptotic pressure values, degrees of non-linearity, time-constants, and/or solid versus fluid proportional response can yield further viscoelastic metrics, such as to allow a user to compare between healthy and diseased states, monitor disease progression, and provide differential diagnosis.

The present inventors have recognized, among other things, that there is a need in the art for apparatus and methods that can monitor or assess a patient lung. The apparatus and methods can include control circuitry, such as capable of running software, configured to process a lung biomarker from patient data. Further, the control circuitry can be configured to generate a lung index, such as to characterize a signature feature of the patient lung to monitor or assess the patient lung. In an example, the lung index can be based at least in part on the lung biomarker, such as to characterize the patient lung.

This summary is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an example of an apparatus, such as to sense an indication of pressure evolution in a patient lung.

FIG. 2 shows an example mouthpiece including an optional volumetric inflator.

FIG. 3 shows an example P-T curve.

FIG. 4 shows an example method for using an apparatus to monitor a patient, such as to monitor a lung condition of the patient.

FIG. 5 shows an example computing machine.

### DETAILED DESCRIPTION

Pulmonary monitoring can be described as a method to track the health of a patient lung, such as by at least one of tracking, charting, or checking performance of lung function over time. In an example, pulmonary monitoring can be used to identify a change in a physiological parameter of the patient lung. A physiological parameter of the patient lung can include any parameter that can describe a characteristic of the patient lung, such as a viscoelastic characteristic of a patient lung. An indication of the physiological parameter can be represented by patient data, such as data collected from the patient with a written questionnaire or measured from the patient with a sensor.

A change in a physiological parameter can indicate at least one of an onset of a lung condition, such as when a value of die physiological parameter deviates from a “normal” patient value of the physiological parameter, or a change in patient lung function, such as indicative of progression of an abnormal lung condition. In an example, a “normal” lung condition can include a state of a patient lung where a medical professional would not recommend a therapeutic intervention, such as based on a physiological parameter of the patient lung. In an example, an “abnormal” lung condition can include a state of a patient lung where a medical professional would recommend a therapeutic intervention, such as based on a physiological parameter of the patient lung. In an example, the term “lung condition” can refer to either of a “normal” or “abnormal” lung condition, such as based on. the context in which the term is used.

In an example, pulmonary monitoring can include at least one of early detection of a lung condition, diagnosis of a lung condition, or assessing patient response to a treatment regimen, such as for a lung condition. In an example, a treatment regimen can include an intervention, such as removing the patient from a toxin/allergen environment, to understand the effect the environment on the patient, such as to improve patient lung health and mitigate further damage.

Pressure evolution can be described as a change, such as a change in pressure experienced in a patient respiratory tract or a patient lung over time. In an example, pressure evolution can also refer to at least one of temporal pressure evolution, temporal pressure dissipation, or temporal pressure relaxation, such as experienced in at least a portion of the patient respiratory tract or the patient lung. Pressure evolution can be understood as stress-relaxation response of the tissue, such as the stress-relaxation response of the lung to an inspiratory breath held by the patient for a period of time. The pressure evolution response can include an indication of a physiological parameter, such as related to the patient lung. In an example, the pressure evolution response can characterize the physiological parameter, such as at least one of a change in patient lung pressure or a change in distance (e.g., displacement) between two landmarks on the patient lung. An indication of pressure evolution response can be obtained from patient data, such as patient data related to the physiological parameter sensed from the patient with a sensor. In an example, the indication of pressure evolution response can be related to a dynamic (or flow) measurement of fluid, such as fluid flow into (e.g., inspiration) or out of (e.g., expiration) the patient lung. In an example, the indication of pressure evolution can be related to a static (or non-flow) measurement of fluid, such as an indication associated with pressure evolution from a patient holding an inhaled breath (e.g., a held breath).

Respiration can include a physiological process where an organism, such as a human, can extract oxygen from the environment, such as by inhaling a gas mixture including ambient air into a lung of the human. In an example, respiration can include receiving a breath, such as can include the act of breathing. Breathing can include a passive process, such as at least one of inspiration or expiration through a combination of at least one of relaxation of the respiratory muscles or the elastic recoil of the lungs and thorax. The volume of the lungs can dictate the inspiratory volume (e.g., the inhaled breath) a patient can receive within the lungs, such as inspiratory volume can be related to at least one of the elasticity of lung tissue or the volume of the thoracic cavity.

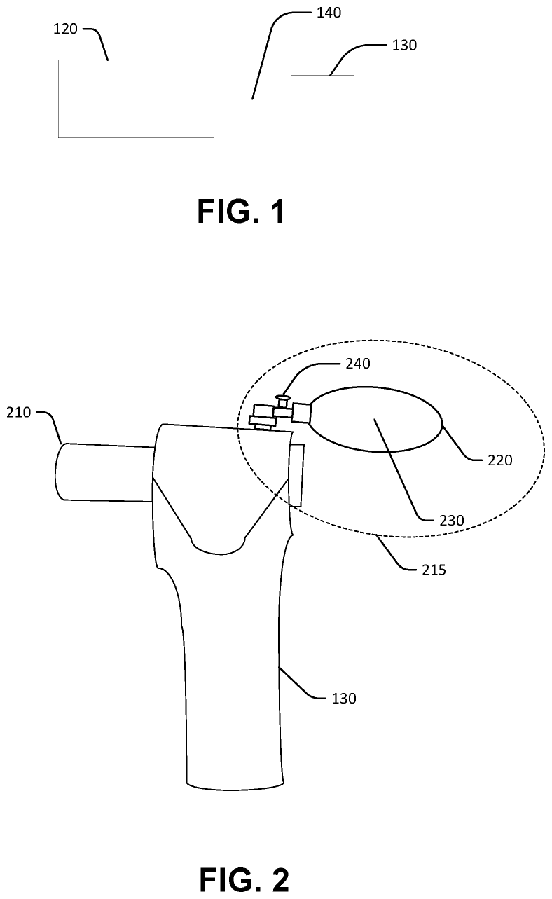

FIG. 1 shows an example of an apparatus 100, such as to sense an indication of pressure evolution response in a patient lung. The apparatus 100 can include control circuitry 120 and, optionally, a sensor 130, such as connected to the control circuitry 120 with a connector 140. In an example, the apparatus 100 can include control circuitry 120 configured to receive patient data, such as patient data related to a physiological parameter of a patient including an indication of pressure evolution front the patient, and process the patient data, such as to process the indication of pressure evolution response to form a lung biomarker. In an example, the apparatus 100 can include the sensor 130, such as the sensor 130 configured to sense patient data, such as an indication of the patient lung, including an indication of pressure evolution response from the patient, and the control circuitry 120, such as configured to receive and process the indication of pressure evolution from the sensor 130.

The control circuitry 120 can facilitate and coordinate operation of the apparatus 100. In an example, the control circuitry 120 can be coupled to, such as in at least one of mechanical or electrical communication with, the sensor 130. In an example, mechanical communication can include the apparatus 100, such as where the sensor 130 can be attached to the control circuitry 120. In an example, electrical communication can include the transfer of patient data sensed by the sensor 130, such as representing an indication of pressure evolution response, to the control circuitry 120, such as through the connector 140. In an example, the connector 140 can include at least one of a wired connection, such as patient data can be transferred from the sensor 130 to the control circuitry 120 with a wire, or a wireless connection, such as electronic hardware utilizing a Wi-Fi or other wireless protocol to transfer data from the sensor 130 to the control circuitry 120.

The control circuitry 120 can include an input device 512, such as configured to allow a user to interact with the apparatus 100. In an example, a user can include at least one of a patient, a patient caregiver, a health professional, or a non-person, such as a computing machine 500 or a data storage device.

The input device 512 can be configured to receive patient data. In an example, the input device 512 cart include a graphical user interface (GUI), such as configured to receive patient data from the user including information related to at least one of basic system functionality (e.g., start/stop of apparatus 100), an indication of user preference, such as a level of patient comfort during operation of the apparatus 100, or an indication of patient health history. In an example, the input device 512 can include an electronic interface, such as to receive patient data from at least one of a sensor 130 configured to contemporaneously sense patient data from the patient and transfer the patient data to the input device 512, or a data storage device, such as configured to transfer patient data previously sensed from the patient and stored on the data storage device to the control circuitry 120.

The control circuitry 120 can include a processing module, such as a programmable central processing unit (CPU). The CPU can execute an instruction, such as one or more instructions, to implement a method of using the apparatus 100, such as to compare patient data as described elsewhere in this application. In an example, the CPU can be a component of a computing machine, such as a computing machine 500.

The CPU can be configured to process received patient data, such as patient data received from the input device 512, to form an indication of a lung biomarker. An indication of a lung biomarker can include at least one of a Group 1 lung biomarker, such as an indication of patient health history, a Group 2 lung biomarker, such as an indication of a dynamic characteristic of the patient lung, or a Group 3 lung biomarker, such as an indication of a viscoelastic characteristic of the patient lung.

The CPU can be configured to process an indication, such as an indication of a lung biomarker, or to generate an indication of an index, such as a lung index configured to characterize the state or condition of the patient lung. A lung index can include a composite indicator of patient lung condition, such as described elsewhere in this application.

The control circuitry 120 can include a storage device 522 to monitor and record patient data, such as an indication of at least one of a lung biomarker or a lung index. The patient data, can be monitored and recorded by the storage device 522 for a period of time, such as for a period of seconds, minutes, hours, days, years, or for the lifetime of the patient.

The control circuitry 120 can include a power source, such as to supply electrical energy to the apparatus 100. In an example, the power source can include at least one of a battery, such as a lithium ion battery, or a transformer, such as to receive power from a wall outlet for use in the apparatus 100 at a specified voltage and current.

### Biomarkers

A biological marker (or a biomarker) can include an indicator, such as a subjective or objective indication of patient health. In an example, a biomarker can include an indication of patient lung health, such as a lung biomarker selected as an indication of the health condition of a patient lung at a selected point in time. The lung biomarker can be processed front patient data sensed from a patient at periodic intervals, such as at least at one of daily, weekly, monthly, or yearly intervals. The lung biomarker can be compared, such as to monitor the patient lung condition or to enable a patient diagnosis based upon objective criteria. In an example, the patient lung biomarker can be compared to patient data, such as the patient lung biomarker can be compared to patient lung biomarker data collected previously from the same patient to monitor progression of a lung condition, or population data, such as the patient lung biomarker can be compared to lung biomarker data collected from others different than the patient, such as to provide an indication of at least one of patient prognosis for a treatment regimen or epidemiological data for public health assessment.

A lung biomarker can include a Group 1 lung biomarker, such as health history data of a patient. Health history data can inform a patient health assessment, such as to provide context for monitoring of patient lung health over an extended period of time.

Health history can include an indication of an objective diagnostic measure, such as to characterize the patient condition. An objective diagnostic measure can include at least one of height, weight, blood-oxygen level, or systemic blood pressure including systolic and diastolic blood pressure. In an example, an objective diagnostic measure can also include an indication of one or more metrics associated with the use of spirometry and imaging, such as to stratify classes of patients including COPD patients, an indication of a Tiffeneau-Pinelli index (e.g., FEV1 ratio), an indication of positive end-expiratory pressure (e.g., PEEP), or an indication of patient respiratory tidal volume.

Health history data can include an indication of a subjective diagnostic measure, such as to characterize the patient condition. A subjective diagnostic measure can include at least one of a patient complaint, such as a patient statement regarding past or present general health condition or past or present lung condition. In an example, a statement of health condition can include an observation, such as “shortness of breath”, “persistent cough”, or “dizziness when I stand”. A subjective diagnostic measure can include the timing of the patient complaint, such as whether the patient complaint pertains to an acute event lasting hours or days, or a chronic event lasting days, weeks, months, or years. A subjective diagnostic measure can include an observation of the patient by another user, such as a medical professional. In an example, an observation can include a present-sense observation of a patient health condition, such as a user observation that an observed patient “is wheezing” or “appears to be in pain” during physical exertion.

Health history data can be collected, such as with at least one of a written questionnaire answered by the patient or a verbal interview, such as with a health professional.

Health history data can be processed, such as to prepare the data for further analysis. In an example, the health history data can be stored, such as in at least one of an analog format including paper records or a digital format including an electronic record. In an example, health history data can be organized to allow for an objective scale to be applied to the health history data for inclusion or use in another metric, such as a lung index metric. An objective scale can include a numerical scale, such as a numerical scale to quantify (or normalize) a patient response for comparison with another patient response. In an example, a numerical scale including delineations of “1”, “2”, “3”, “4”, and “5” can be applied to a patient response to the question, “how are you feeling today?”. For example, a patient response of “feeling bad” can be assigned a value of “1”, such as to indicate a lower bound of patient condition, a patient response of “feeling good” can be assigned a value of “5”, such as to indicate an upper bound of patient condition, and a patient response other than “feeling bad” or “feeling good” can be assigned a value between “1” and “5”, such as to locate the response relative to the lower and upper patient condition bounds.

A lung biomarker can include a Group 2 lung biomarker, such as a dynamic lung characteristic of the patient.

A dynamic lung characteristic can include a dimensional measurement of the lung that can change over time, such as with patient respiration. A dynamic lung characteristic can include an indication of patient lung displacement, such as an indication of a change in displacement between two landmarks on the patient lung. A lung landmark can include any selected location on the patient lung that can be monitored, such as located or “tracked”, over a period of time, such as with a sensor 130. In an example, the indication of a change in displacement can include at least one of an indication of a change in distance, an indication of a change in velocity, or aa indication of a change in acceleration. A dynamic lung characteristic can include an indication of patient lung volume, such as an indication of a change in displacement between two or more landmarks on the patient lung. In an example, the indication of a change in volume can include at least one of an indication of a change in distance between the two or more landmarks defining the volume, an indication of a change in velocity of the two or more landmarks, or an indication of a change in acceleration of the two or more landmarks.

The dynamic lung characteristic can be collected or otherwise received from the patient, such as with a sensor 130 integrated into a sensor system.

The sensor 130 can include a pressure sensor, such as a pressure sensor system. In an example, the sensor can include a mouthpiece 210 with an integrated pressure sensor, such as described elsewhere in this application. The pressure sensor can be configured to sense an indication of the patient lung, such as an indication of pressure evolution in the patient lung. In an example, the indication of the patient lung can include a pressure-time (or P-T) curve, such as related to pressure evolution in the lung associated with at least one of a dynamic (or flow) measurement of pressure, such as during patient respiration, or a static (or non-flow) measurement of pressure, such as related to a patient held breath for a period of time.

FIG. 2 shows an example sensor 130, such as a mouthpiece 210 including an optional volumetric inflator 215. The pressure sensor can be included in or attached to the mouthpiece 210, such as to sense pressure evolution in the patient mouth or respiratory tract. In an example, the mouthpiece 210 can be configured or shaped, such as to locate the pressure sensor at a selected location in the patient mouth, respiratory tract, or lung.

The volumetric inflator 215 can optionally be attached to the mouthpiece 210, such as to introduce a selected volume of air into the patient respiratory tract, such as to sense an indication of pressure evolution in the patient lung subject to a known inflation volume. The volumetric inflator 215 can be used optionally with the mouthpiece 210, such as a surrogate ventilation device when patient volume inspiration effort is insufficient to sense an indication of pressure evolution in the patient lung. The volumetric inflator 215 can include at least one of a balloon 220, such as a closed membrane configured to separate a volume 230 enclosed by the membrane from the surrounding atmosphere, and a relief valve 240. In an example, the volumetric inflator 215 can include a bellows device. In an example, the volumetric inflator 215 can be located in communication with the patient mouth, such as in communication with the mouthpiece 210, and compressed, such as to by force fluid from the volume 230 into the patient lung to provide positive pressure ventilation and expand the patient lung. Expansion of the patient lung can assist in sensing a patient data, such as an indication of pressure evolution in the patient lung. In an example, the fluid in the volume 230 can include a gaseous fluid, such as at least one of ambient air or a fluid with a composition other than ambient air, such as a composition selected to treat the patient lung or assist in sensing an indication of pressure evolution in the patient lung. The relief value 240 can be configured to close during compression of the volumetric inflator 215, such as to force fluid into the patient lung, and open during rarefaction of the volumetric inflator 215, such as to allow a fluid to flow into the volume 230 including from the surrounding atmosphere, such as to prevent negative pressure ventilation of the patient.

The sensor 130 can include at least one of an ultrasonic sensor, such as an ultrasonic sensor system associated with use in sonography, or an X-ray sensor, such as an X-ray sensor system associated with radiography. The ultrasound sensor or the X-ray sensor can be configured to sense patient data, such as an indication of lung displacement including a change of distance between two landmarks on the patient lung. The indication of displacement can be related to pressure evolution in the lung associated with at least one of a dynamic (or flow) measurement of pressure during patient inspiration or expiration or a static (or non-flow) measurement of pressure. In an example, the indication of lung displacement can be combined with other information, such as an estimate of patient lung elasticity, to estimate a change in lung pressure with respect to time, such as to generate a P-T curve or similar metric.

The sensor 130 can include an MRI sensor, such as an MRI sensor system associated with use in medical imaging. The MRI sensor can be configured to sense an indication of the patient lung, such as an indication of displacement including a change of distance between two landmarks on the patient lung. The indication of displacement can be related to pressure evolution in the lung associated with at least one of a dynamic (or flow) measurement of pressure during patient inspiration or expiration or a static (or non-flow) measurement of pressure. In an example, the indication of displacement can be combined with other information, such as an estimate of patient lung elasticity, to estimate a change in lung pressure with respect to time, such as to generate a P-T curve or similar metric, or a change in lung volume with respect to time.

The dynamic lung characteristic data can be processed, such as to prepare the data for further analysis. In an example, dynamic lung characteristic data can be stored, such as in at least one of an analog format including paper records or a digital format including an electronic record, such as to a storage device 522. In an example, dynamic lung characteristic data can be correlated, such as the dynamic lung characteristic data can be considered as an indication of a viscoelastic characteristic of the lung. For example, an indication of displacement, such as a change in displacement, between two landmarks on a patient lung due to pressure evolution, such as during a held breath, can be correlated to a characteristic of the patient lung, such as a viscoelastic characteristic of the patient lung. In an example, one or more dynamic lung characteristic can be organized for inclusion or use in another metric, such as a lung index metric.

A lung biomarker can include a Group 3 lung biomarker, such as a viscoelastic characteristic of a patient lung. A viscoelastic characteristic can describe the property of tissue, such as at least one of elastic tissue behavior or viscous tissue behavior. In an example, a Group 3 lung biomarker can include a viscoelastic characteristic, such as a patient lung viscoelastic parameter (PLVP) including a signature viscoelastic feature.

Patient data can be collected from a patient, such as to characterize a patient physiological parameter. In an example, patent data can be collected by a survey, such as by asking a question of the patient and recording the patient response.

In an example, patient data can be collected with a sensor 130, such as with a sensor 130 integrated into a sensor system as described elsewhere in this application. In an example, the sensor 130 can include at least one of a pressure sensor system, an ultrasound system, an MRI system, or an X-ray system.

Patient data collected with the sensor 130, such as a pressure sensor system, can include an indication of a physiological parameter, such as an indication of a change in patient lung pressure related to a held breath sensed in a patient over a period of time. In an example, an indication of change in patent lung pressure over time can include a pressure versus time (or P-T) curve.

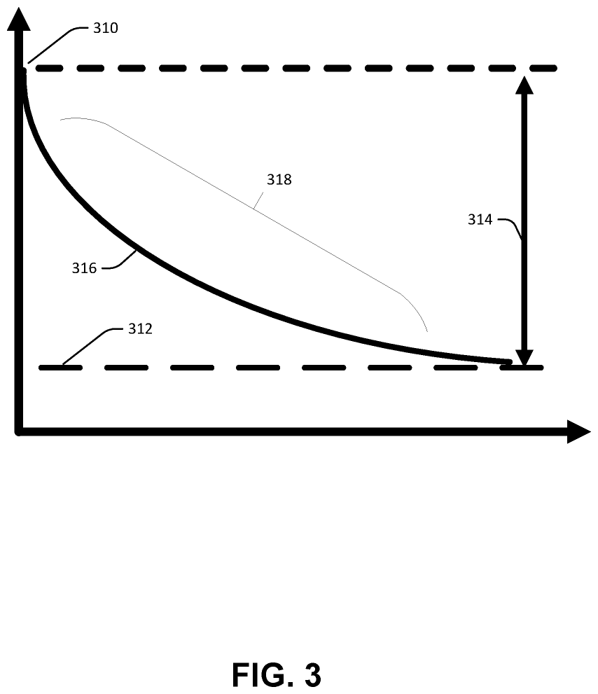

FIG. 3 shows an example P-T curve, such as representing pressure evolution in a patient respiratory tract. In an example, the horizontal axis can represent time and the vertical axis can represent pressure, such as lung pressure magnitude. The P-T curve can be characterized by an indication of a physiological parameter, such as at least one of an indication of peak pressure 310 (or Pp), an indication of asymptotic pressure 312, an indication of fractional relaxation 314, an indication of a time-constant 316, or an indication of degree of model non-linearity 318.

Patient data can be “reduced” or curvefit with a mathematical (or math) model, such as to generate a value for one or more model parameter variable (MPV) to characterize the patient data. A math model can be used to define or describe a lung biomarker, such as an MPV value to characterize at least one of a Group 2 lung biomarker or a Group 3 lung biomarker. An MPV can include a variable in a math model, such as the value of the variable that can define a curve to curvet the patient data. ln an example, a math model can include a rheological math model including at least one of a Fractional Standard Linear Solid model, a Maxwell model, or a Kelvin model. In an example, an indication of an MPV value can represent an indication of a PLVP, such as an indication of patient lung viscoelasticity.

In an example, an exponential decay model, such as a linear first-order ordinary differential equation defined by a time constant parameter, can be applied to patient data. The collected patient data can be processed, or otherwise curvefit to approximate a “best-fit” curve to identify a value for the time constant parameter, such as to characterize the collected patient data. In an example, a best-fit characterization can include identifying a value for an MPV, such as an MPV selected to minimize error between the mathematical model and the collected data, such as using a least squares error metric. The value of the time constant parameter, such as resulting from curve fitting the mathematical model to the collected patient data, can represent a PLVP, such as an indication of patient lung viscoelasticity estimated from the exponential decay model. Referring again to FIG. 3, the PLVP, such as estimated from the exponential decay model, can include a viscoelastic characteristic of the patient lung, such as to characterize the viscoelastic characteristic of the “bulk” or “whole organ”.

A PLVT can include an indication of peak pressure (Pp), such as an inspiratory peak pressure associated with a patient held breath in a P-T curve. In an example, Pp can be increased for a patient, such as with the use of the optional volumetric inflator 215.

A PLVP can include an indication of fractional relaxation of the patient lung, such as an indication of fractional relaxation formed from information in a P-T curve. The fractional relaxation can include a ratio, such as the ratio of peak pressure to a selected asymptotic value. In an example, the selected asymptotic value can include the sensed pressure from the P-T curve, such as at a selected time after peak pressure.

The indication of fractional relaxation can be influenced by the data examined, such as the value of an indication of fractional relaxation can be affected by the portion of the P-T curve examined during a curve-fit. In an example, a value of an indication of fractional relaxation can be estimated at a selected time, such as one or more selected times, associated with the P-T curve measurement, such as to obtain a value for an indication of fractional relaxation at the one or more selected times. For example, a user can estimate a value for an indication of fractional relaxation at a selected time of at least one of 1 second after peak pressure (Pp), at 5 seconds after Pp, at 10 seconds after Pp, or at 20 seconds after Pp, such as to characterize the patient lung for use as an indication of a lung biomarker.

A PLVP can include an indication of percent relaxation of the patient lung, such as a percentage indication of fractional relaxation. In an example, a value for percent relaxation can be formed by multiplying fractional relaxation by 100, such as to generate a percentage level of peak pressure to the selected asymptotical value.

A PLVP can include an indication of a time constant, such as a time constant associated with an exponential decay model. In an example, a math model, such as a fractional standard linear solid model, can be used to identify an indication of a lung biomarker, such as to characterize a patient P-T curve with at least one of a “solid-like” contribution metric and a “fluid-like” contribution metric. In an example, the contribution metrics can be characterized with a standard exponential model, such as a model described with a model parameter including at least one of a base, an exponent (e.g., a power of the base), or a coefficient (e.g., a gain applied to the base), where a value of the model parameter can serve as an indication of a lung biomarker.

A PLVP can include an indication of non-linearity, such as for patient data where least squares error associated with a linear math model can be reduced by applying a non-linear math model. In an example, the example of the indication of fractional relaxation influenced by the data examined (see above) can be described by an exponential decay model characterized by a non-linear time constant, such as an indication of non-linearity can include a metric to describe the non-linearity of the time constant.

### Lung Index

A lung biomarker, such as one or more lung biomarkers can be combined, such as to form a lung index. A lung index can include a composite indicator, such as a combination, at least in part, of one or more lung biomarkers that can form an improved monitoring or diagnostic tool as compared to the constituent lung biomarkers alone, such as to characterize the patient lung.

One or more lung biomarkers can be collected, such as into a group of lung biomarkers that have a common characteristic. As such, a set of appropriately grouped biomarkers can be used, such as by a medical professional to predict or diagnose a potential lung condition in a patient.

In an example, a Group 1 lung biomarker, such as describing a patient health history, can be considered at least one of a present or lagging indicator for a lung condition. For example, an objective diagnostic measure, such as blood-oxygen level, or a subjective diagnostic measure, such as a patient statement of present health condition, can indicate the presence or progression of a lung condition, such as in a patient with a history of a lung condition.

In an example, a Group 2 lung biomarker, such as describing a dynamic lung characteristic of a patient lung, can be considered a present or leading indicator for a lung condition. Changes in lung displacement, such as between two lung landmarks, or changes in lung volume can, in some cases, signal the presence of a lung condition. For example, a decrease in lung, displacement or lung volume, such as signaled by patient exercise intolerance or direct measurement of the patient with a sensor 130, can indicate the presence of a potential lung condition, such as in a sedentary patient.

In an example, a Group 3 lung biomarker, such as describing a viscoelastic character of a patient lung, can be considered a present or a leading indicator for a lung condition. Subtle changes in viscoelastic behavior of patient lung tissue at the molecular level can, in some cases, anticipate pathological progression of a lung condition. For example, a decrease in patient lung viscoelasticity, such as compared to the general population, can indicate the presence of a potential lung condition, such as in an asymptomatic patient.

The lung index can include, at least in part, a lung biomarker, such as a lung biomarker from at least one of the Group 1 lung biomarker, the Group 2 lung biomarker or the Group 3 lung biomarker. In an example, the lung index can include, at least in part, a lung biomarker selected from each of the Group 1 lung biomarker and the Group 2 lung biomarker. In an example, the lung index can include, at least in part, a lung biomarker selected from each of the Group 1 lung biomarker and the Group 3 lung biomarker. In an example, the lung index can include, at least in part, a lung biomarker selected from each of the Group 2 lung biomarker and the Group 3 lung biomarker. In an example, the lung index can include, at least in part, a lung biomarker selected from each of the Group 1 lung biomarker, the Group 2 lung biomarker, and the Group 3 lung biomarker.

### Methods

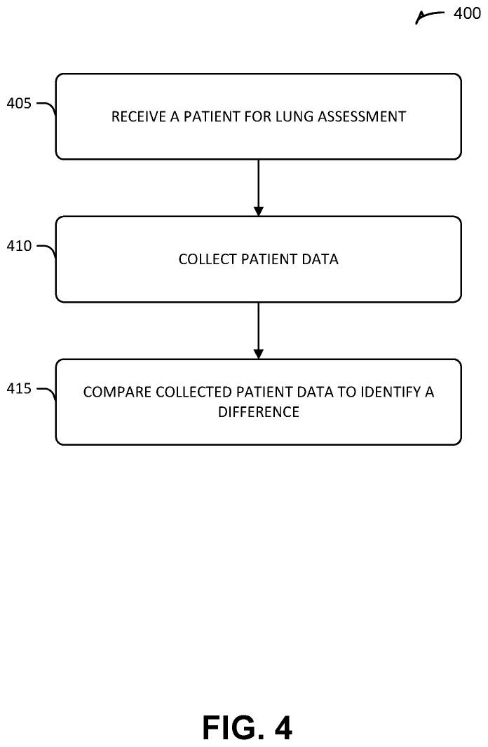

FIG. 4 shows an example method 300 for using an apparatus, such as the apparatus 100, to monitor a patient, such as to monitor a lung condition of the patient. The apparatus 100 can include control circuitry 120, such as control circuitry configured to receive patient data related to a patient and process the received patient data, such as to form at least one of a lung biomarker or a lung index. A method, such as the example method 400, can be embodied in one or more data structures or instructions, such as implemented on a computing machine 500. In an example, patient data can include an indication of a physiological parameter, such as an indication of a lung biomarker from the patient, or an indication of patient health history.

At 405, a patient can be received, such as by a medical professional to assess the patient lung. Receiving a patient can include at least one of examining the patient, such as to screen the patient for a lung condition, diagnosing the patient, such as to deliver a recommendation as to the probability of a lung condition based on data available to the medical professional, or monitoring the patient, such as to assess the progression of a previously diagnosed lung condition by comparison of present patient data, such as an indication of a present lung index score, to previous patient data, such as an indication of a lung index score from a previous encounter.

At 405, patient data can be collected, such as for use as a lung biomarker. Collecting patient data can include as least one of receiving contemporaneous patient data, such as patient data collected from the patient upon receiving the patient, or receiving stored patient data, such as patient data collected prior to receiving the patient.

Collecting patient data can include interviewing the patient, such as to collect health history data from the patient. In an example, collecting patient data can include collecting Group 1 lung biomarker data from the patient.

Collecting patient data can include processing collected health history data, such as to form a lung biomarker. In an example, processing can include applying an objective scale to health history data, such as a numerical scale of 1 to 5 to form an indication of a lung biomarker. In an example, processing health history data can include forming a lung index, such as at least in part from an indication of the lung biomarker.

Collecting patient data can include sensing an indication of a dynamic lung characteristic from the patient, such as a dimensional measurement of the lung that can change over time. In an example, collecting patient data can include collecting Group 2 lung biomarker data from the patient.

Collecting patient data can include processing an indication of a dynamic lung characteristic from the patient, such as to form a lung biomarker. In an example, processing patient data can include estimating the lung biomarker, such as from a dynamic lung characteristic. In an example, processing an indication of a dynamic lung characteristic can include correlating an indication of a dynamic lung characteristic, such as an indication of a change in distance between two landmarks on a patient lung due to pressure evolution during a held breath, with a characteristic of the patient lung, such as a viscoelastic characteristic of the patient lung. In an example, processing patient data can include forming a lung index, such as at least in part from an indication of a dynamic lung characteristic.

Collecting patient data can include sensing an indication of a viscoelastic characteristic from a patient lung, such as to form a lung biomarker. In an example collecting patient data can include collecting Group 3 lung biomarker data from the patient.

Collecting patient data can include processing an indication of a viscoelastic characteristic from a patient lung, such as to form the lung biomarker. In an example, processing an indication of a viscoelastic characteristic can include generating a model parameter variable (MPV) from a math model, such as to estimate an indication of a lung biomarker. In an example, the MPV can include a patient lung viscoelastic parameter (PLVP). In an example, processing patient data can include forming a lung index, such as at least in part from an indication of a viscoelastic characteristic of the patient lung.

At 415, patient data can be compared, such as to identify a difference between as first patient data set and a second patient data set. Comparing patient data can allow a user, such as a medical professional, to observe a change in one or more lung biomarkers, such as to indicate the presence of a lung condition in the patient.

Comparing patient data can include forming a metric, such as a composite metric to characterize a patient lung condition based at least in part on one or more lung biomarkers. In an example, the composite metric can include a lung index, such as at least one of selected lung biomarkers or an arrangement of patient data configured to indicate a patient risk for a lung condition, such as indicative of an increased or decreased risk of the presence of aa lung condition.

Comparing patient data can include comparing data from the same patient, such as to form a first example of a lung index. In an example, a first patient data set, such as a collected from a patient at a first time, and a second patient data set, such as collected front the patient at a second time, can be compared, such as to identify a change in one or more lung biomarkers that can be indicative of patient lung health. For example, a user can compare a baseline biomarker value, such as collected and processed from a previous visit of the patient to the medical professional, with a subsequent biomarker value, such as collected and processed during a visit contemporaneous with the comparison to the baseline biomarker value, such as to indicate the presence of a lung condition in the patient.

Comparing patient data can include comparing a patient data set to a “nominal” patient data set, such as to form a second example of a lung index. A nominal patient data set can include a composite patient data set, such as a data set formed from epidemiological data and configured to represent the characteristics of a nominal (or average) patient. In an example, a first patient data set, such as collected from a patient, and a second patient data set, such as a nominal patient data set, can be compared, such as to identify a deviation in the first patient data set with respect to the nominal patient data set, such as to indicate the presence of a lung condition in the patient.

Comparing patient data can include applying a mathematical operation to a biomarker, such as one or more biomarkers in a patient data set, such as to form a third example of a lung index. In an example, a mathematical operation can include at least one of addition, subtraction, multiplication, division, or a combination of operations.

Inspection of individual lung biomarkers, such as independent inspection for changes in at least one of a present indicator (or Group 2 lung biomarker) or a leading indicator (or Group 3 lung biomarker), can result in an indefinite finding (e.g., weak signal) of a lung condition, such as when the changes are of small magnitude as compared to the lung biomarker level. However, a mathematical combination of individual lung, biomarkers can magnify information contained within the one or more indication of present and leading indicators, such as to clarify a finding (e.g., strong signal) of aa lung condition. In an example, dividing a Group 3 lung biomarker value (leading indicator) by a Group 2 lung biomarker value (present indicator) can result in a ratio, such as an example of a fourth lung index. For example, a fourth lung index greater than 1, such as indicating a greater difference between a first and second Group 3 lung biomarker than between a first and second Group 2 lung biomarker, can indicate an increased risk for a lung condition, such as a lung condition in an asymptomatic patient.

Comparing patient data can include diagnosing a patient lung condition, such as in an asymptomatic patient. The use of at least one of a leading indicator, such as a Group 3 lung biomarker, or a present indicator, such as a Group 2 lung biomarker or a Group 1 lung biomarker, can improve diagnosis of a lung condition in a patient. Correlating experimental data, such as from a clinical trial, with a selected combination of one or more lung biomarkers, such as forming a lung index, can assist a medical profession in patient diagnosis, such as to distinguish a first suspected lung condition from a second suspected lung condition. In an example, diagnosis of a lung condition in an asymptomatic patient can afford options to the patient, such as to initiate a therapeutic regimen to treat the lung condition.

### Computing Machine

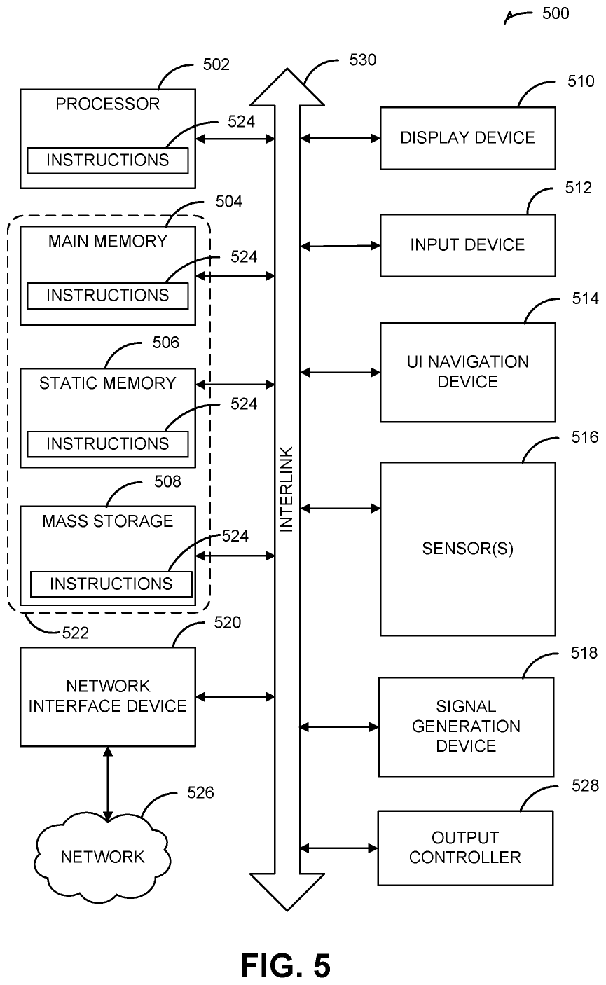

FIG. 5 illustrates a block diagram of an example machine 500 upon which any one or more of the techniques (e.g., methodologies) discussed herein may perform. In an embodiment, the apparatus 100 communicates with the machine 500 (e.g., a server machine) which may be used to receive patient data, such as from the sensor 130, process patient data, such as to form at least one of a lung biomarker or a lung index, and execute the trained models and provide the motion controls based on inferred intended movement, according to the contextual data. The machine 500 may be a local or remote computer, or processing node in an on-the-go (OTG) device such as a smartphone, tablet, or wearable device. The machine 500 may operate as a standalone device or may be connected (e.g., networked) to other machines. In an embodiment, the machine 500 may be directly coupled or be integrated with the apparatus 100. In a networked deployment, the machine 500 may operate the capacity of a server machine, a client machine, or both in server-client network environments. In an example, the machine 500 may act as a peer machine in peer-to-peer (P2P) (or other distributed) network environment. The machine 500 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuitry is a collection of circuits implemented in tangible entities that include hardware (e.g., simple circuits, gates, logic, etc.). Circuitry membership may be flexible over time and underlying hardware variability. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, the computer readable medium is communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time.

Machine (e.g., computer system) 500 may include a hardware processor 502 (e.g., a central processing unit (CPU), a graphics processing unit (GPU), a hardware processor core, or any combination thereof), a main memory 504 and a static memory 506, some or all of which may communicate with each other via an interlink (e.g., bus) 530. The machine 500 may further include a display unit 510, an input device 512, such as at least one of a keyboard, a graphical user interface (GUI), or an electronic interface, such as to receive a signal from a sensor, and a user interface (UI) navigation device 514 (e.g., a mouse). In an example, the display unit 510, input device 512 and UI navigation device 514 may be a touch screen display. The machine 500 may additionally include a storage device (e.g., drive unit) 522, a signal generation device 518 (e.g., a speaker), a network interface device 520, and one or more sensors 516, such as a sensor 130, a global positioning system (GPS) sensor, compass, accelerometer, or other sensor. In an example, sensors 516 including sensor 130 may include wearable or non-wearable sensors, such as described elsewhere in this application. The machine 500 may include an output controller 528, such as a serial (e.g., universal serial bus (USB), parallel, or other wired or wireless (e.g., infrared (IR), near field communication (NFC), etc.) connection to communicate or control. One or more peripheral devices (e.g., a printer, card reader, etc.).

The storage device 522 may include a machine readable medium 508 on which is stored one or more sets of data structures or instructions 524 (e.g., software) embodying or utilized by any one or more of the techniques or functions described herein. The instructions 524 may also reside, completely or at least partially, within the main memory 504, within static memory 506. or within the hardware processor 502 during execution thereof by the machine 500. In an example, one or any combination of the hardware processor 502, the main memory 504, the static memory 506, or the storage device 516 may constitute machine readable media.

While the machine readable medium 508 is illustrated as a single medium, the term “machine readable medium” may include a single medium or multiple media (e.g., a centralized or distributed database, or associated caches and servers) configured to store the one or more instructions 524.

The term “machine readable medium” may include any medium that is capable of storing, encoding, or carrying instructions for execution by the machine 500 and that cause the machine 500 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 524 may further be transmitted or received over a communications network including an interlink 530 using a transmission medium via the network interface device 520 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.X family of standards, peer-to-peer (P2P) networks, among others. In an example, the network interface device 520 may include one or more physical jacks (e.g., Ethernet, coaxial, or phone jacks) or one or more antennas to connect to the communications network 526. In an example, the network interface device 520 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 500, and includes digital or analog communications signals or other intangible medium to facilitate communication of such software.

The techniques described herein are not limited to any particular hardware or software configuration; they may find applicability in any computing, consumer electronics, or processing environment. The techniques may be implemented in hardware, software, firmware or a combination, resulting in logic or circuitry which supports execution or performance of embodiments described herein.

For simulations, program code may represent hardware using a hardware description language or another functional description language which essentially provides a model of how designed hardware is expected to perform. Program code may be assembly or machine language, or data that may be compiled or interpreted. Furthermore, it is common in the art to speak of software, in one form or another as taking an action or causing a result. Such expressions are merely a shorthand way of stating execution of program code by a processing system which causes a processor to perform an action or produce a result.

Each program may be implemented in a high-level procedural, declarative, or object-oriented programming language to communicate with a processing system. However, programs may be implemented in assembly or machine language, if desired. In any case, the language may be compiled or interpreted.

Program instructions may be used to cause a general-purpose or special-purpose processing system that is programmed with the instructions to perform the operations described herein. Alternatively, the operations may be performed by specific hardware components that contain hardwired logic for performing the operations, or by any combination of programmed computer components and custom hardware components. The methods described herein may be provided as a computer program product, also described as a computer or machine accessible or readable medium that may include one or more machine accessible storage media having stored thereon instructions that may be used to program a processing system or other electronic device to perform the methods.

Program code, or instructions, may be stored in, for example, volatile or non-volatile memory, such as storage devices or an associated machine readable or machine accessible medium including solid-state memory, hard drives, floppy-disks, optical storage, tapes, flash memory, memory sticks, digital video disks, digital versatile discs (DVDs), etc., as well as more exotic mediums such as machine-accessible biological state preserving storage. A machine readable medium may include any mechanism for storing, transmitting, or receiving information in a form readable by a machine, and the medium may include a tangible medium through which electrical, optical, acoustical or other form of propagated signals or carrier wave encoding the program code may pass, such as antennas, optical fibers, communications interfaces, etc. Program code may be transmitted in the form of packets, serial data, parallel data, propagated signals, etc., and may be used in a compressed or encrypted format.

Program code may be implemented in programs executing, on programmable machines such as mobile or stationary computers, personal digital assistants, smart phones, mobile Internet devices, set top boxes, cellular telephones and pagers, consumer electronics devices (including DVD players, personal video recorders, personal video players, satellite receivers, stereo receivers, cable TV receivers), and other electronic devices, each including a processor, volatile or non-volatile memory readable by the processor, at least one input device or one or more output devices. Program code may be applied to the data entered using the input device to perform the described embodiments and to generate output information. The output information may be applied to one or more output devices. One of ordinary skill in the art may appreciate that embodiments of the disclosed subject matter may be practiced with various computer system configurations, including multiprocessor or multiple-core processor systems, minicomputers, mainframe computers, as well as pervasive or miniature computers or processors that may be embedded into virtually any device. Embodiments of the disclosed subject matter may also be practiced in distributed computing environments, cloud environments, peer-to-peer or networked microservices, where tasks or portions thereof may be performed by remote processing devices that are linked through a communications network.

A processor subsystem may be used to execute the instruction on the machine-readable or machine accessible media. The processor subsystem may include one or more processors, each with one or more cores. Additionally, the processor subsystem may be disposed on one or more physical devices. The processor subsystem May include one or more specialized processors, such as a graphics processing unit (GPU), a digital signal processor (DSP), a field programmable gate array (FPGA), or a fixed function processor.

Although operations may be described as a sequential process, some of the operations may in fact be performed in parallel, concurrently, or in a distributed environment, and with program code stored locally or remotely for access by single or multi-processor machines. In addition, in some embodiments the order of operations may be rearranged without departing from the spirit of the disclosed subject matter. Program code may be used by or in conjunction with embedded controllers.

Examples, as described herein, may include, or may operate on, circuitry, logic or a number of components, modules, or mechanisms. Modules may be hardware, software, or firmware communicatively coupled to one or more processors in order to carry out the operations described herein. It will be understood that the modules or logic may be implemented in a hardware component or device, software or firmware running on one or more processors, or a combination. The modules may be distinct and independent components integrated by sharing or passing data, or the modules may be subcomponents of a single module, or be split among several modules. The components may be processes running on, or implemented on, a single compute node or distributed among a plurality of compute nodes running in parallel, concurrently, sequentially or a combination, as described more fully in conjunction with the flow diagrams in the figures. As such, modules may be hardware modules, and as such modules may be considered tangible entities capable of performing, specified operations and may be configured or arranged in a certain manner. In an example, circuits may be arranged e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a machine-readable medium. In an example, the software, when executed by the underlying hardware of the module, causes the hardware to perform the specified operations. Accordingly, the term hardware module is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured, arranged or adapted by using software the general-purpose hardware processor may be configured as respective different modules at different times. Software may accordingly configure a hardware processor, for example, to constitute a particular module at one instance of time and to constitute a different module at a different instance of time. Modules may also be software or firmware modules, which operate to perform the methodologies described herein.

### Various Notes

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform. methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An apparatus to assess a patient lung, comprising:
control circuitry configured to process a lung biomarker from patient data and generate a lung index based at least in part on the lung biomarker to characterize the patient lung.

2. The apparatus of claim 1, wherein the lung biomarker includes at least in part at least one of a Group 1 biomarker including a patient health history, a Group 2 biomarker including a dynamic lung characteristic, or a Group 3 biomarker including a viscoelastic characteristic of the patient lung.

3. The apparatus of claim 2, wherein the lung biomarker includes an indication of the Group 3 biomarker.

4. The apparatus of claim 2, wherein the lung index includes at least in part at least one biomarker selected from the Group 1 biomarker and at least one biomarker selected from the Group 2 biomarker.

5. The apparatus of claim 2, wherein the lung index includes at least in part at least one biomarker selected from the Group 1 biomarker and at least one biomarker selected from the Group 3 biomarker.

6. The apparatus of claim 2, wherein the lung index includes at least in part at least one biomarker selected from the Group 2 biomarker and at least one biomarker selected from the Group 3 biomarker.

7. The apparatus of claim 2, wherein the lung index includes at least in part at least one biomarker selected from the Group 1 biomarker, least one biomarker selected from the Group 2 biomarker, and at least one biomarker selected from the Group 3 biomarker.

8. The apparatus of claim 1, further comprising a sensor configured to sense an indication of the patient lung.

9. The apparatus of claim 8, wherein the indication of the patient lung includes an indication of a viscoelastic parameter of the patient lung.

10. The apparatus of claim 8, wherein the sensor includes a pressure sensor configured to sense an indication of an inhaled breath.

11. The apparatus of claim 8, wherein the sensor includes an ultrasonic sensor configured to sense an indication of distance between a first location on the patient lung and a second location on the patient lung.

12. The apparatus of claim 8, wherein the sensor includes a magnetic resonance imaging (MRI) system configured to sense an indication of distance between a first location on the patient lung and a second location on the patient lung.

13. The apparatus of claim 7, wherein the sensor includes an X-ray system configured to sense an indication of distance between a first location on the patient lung and a second location on the patient lung.

14. The apparatus of claim 1, wherein the control circuitry includes a central processing unit (CPU) configured to form the lung biomarker from a mathematical model.

15. The apparatus of claim 1, wherein the control circuitry includes a central processing unit (CPU) configured to estimate the lung biomarker at least in part from an indication of lung displacement.

16. A method for pulmonary monitoring, the method comprising:
non-flow measurement of pressure evolution from an individual holding an inhaled breath.

17. The method according to claim 16, further comprising:
determining based on the measured pressure evolution a heath state of lungs of the individual, and wherein the health state of the lungs of the individual includes normal lung function and abnormal lung function.

18. The method according to claim 17, wherein the health state of the lungs of the individual is the abnormal lung function, the method further comprising:
determining between different types of the abnormal lung function.

19. The method according to claim 18, further comprising:
continuously monitoring the abnormal lung function for disease progression.

20. The method according to claim 1 6, wherein the individual holds their breath for as long as possible.

21. The method according to claim 16, further comprising:
analyzing a decrease of pressure over time from the recorded pressure measurements with rheological models to generate a lung biomarker.

22. The method according to claim 21, wherein lung biomarker includes at least one of an indication of peak pressure, an indication of asymptotic pressure, an indication of fractional relaxation, an indication of degrees of non-linearity, an indication of a time-constant, or an indication of solid versus fluid proportional response.

23. The method according to claim 22, wherein the lung biomarker is a biomarker of disease manifestation.

24. The method according to claim 16, wherein the measured pressure evolution of the fixed volume of air is a measurement of non-flow lung properties, the non-flow properties being viscoelasticity defined as the time (viscous) and stretch (elastic) dependency of lung function.

25. A method to assess a patient lung, comprising:
receiving patient for a lung assessment; and
processing a lung biomarker from a first indication of the patient lung to generate a first lung index to characterize the patient lung at a first measurement time.

26. The method of claim 25, comprising processing the lung biomarker from a subsequent indication of the patient lung to generate a subsequent lung index to characterize the patient lung at a subsequent measurement time different from the first measurement time.

27. The method of claim 25, comprising comparing the first lung index to the subsequent lung index to detect a difference between the first lung index and the subsequent lung index.

28. The method of claim 25, comprising comparing a first subsequent lung index to a second subsequent lung index to detect an incremental difference between the first subsequent lung index and the second subsequent lung index.

29. The method of claim 26, comprising diagnosing a lung condition based on the difference between the first lung index and the subsequent lung index.
